(19) United States
(12) Patent Application Publication
(10) Pub. No.: US 2002/0061289 A1
(43) Pub. Date: May 23, 2002

(54) Stem cell-based methods for preventing and treating tumor

(76) Inventor: Bruce M. Boman, Gladwyne, PA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/969,892

(22) Filed: Oct. 2, 2001

Related U.S. Application Data
(60) Provisional application No. 60/238,665, filed on Oct. 4, 2000.

(51) Int. Cl. A61K 38/19; A61K 38/18; A61K 38/17

(52) U.S. Cl. 424/85.1; 514/2

(57) ABSTRACT

Stem cell-based methods and compositions for treating and preventing tumorigenesis are disclosed.

### CROSS REFERENCE TO RELATED APPLICATIONS

This application is a conversion of U.S. Provisional Application Serial No. 60/238,665 (filed Oct. 4, 2000), the disclosure of which is incorporated herein by reference in its entirety for all purposes.

### SUMMARY OF THE INVENTION

The present invention provides methods for preventing or treating tumor in a subject (e.g., human or non-human animals). The methods comprise administration of an effective amount of chemopreventive or therapeutic agents that stop or reduce generation of mutant stem cells in vivo, that reverse progression of premalignant stem cells that have at least one genetic mutation, or that eliminate premalignant stem cells and tumor stem cell populations.

In some methods, the chemopreventive or therapeutic agents act on premalignant stem cells and tumor stem cells via intracellular, intercellular and tissue pathways that modulate the rate of premalignant or tumor stem cell proliferation. In some methods, chemopreventive or therapeutic agents act on premalignant stem cells and tumor stem cells via intracellular, intercellular or tissue pathways that modulate the number of premalignant or tumor stem cells in vivo. In some other methods, the chemopreventive or therapeutic agents act on premalignant stem cells and tumor stem cells via pathways involving growth factors, cytokines, and their receptors including those that function via autocrine, paracrine, and endocrine loops which control premalignant or tumor stem cells. In other methods, the chemopreventive or therapeutic agents act on premalignant stem cells and tumor stem cells via cell-to-cell or cell-to-matrix signaling pathways including epithelial stem cell-epithelial stem cell, epithelial stem cell-to-stromal cell, and stem cell-local extracellular matrix environmental interactions or communications. In some methods, the chemopreventive or therapeutic agents act on premalignant stem cells and tumor stem cells via biological pathways that function during development, which lead to the generation of premalignant or tumor stem cells during the development of a subject. In some other methods, the chemopreventive or therapeutic agents act on premalignant stem cells and tumor stem cells via immunological pathways that control premalignant or tumor stem cells.

### BRIEF DESCRIPTION OF THE DRAWINGS

FIG. 1.

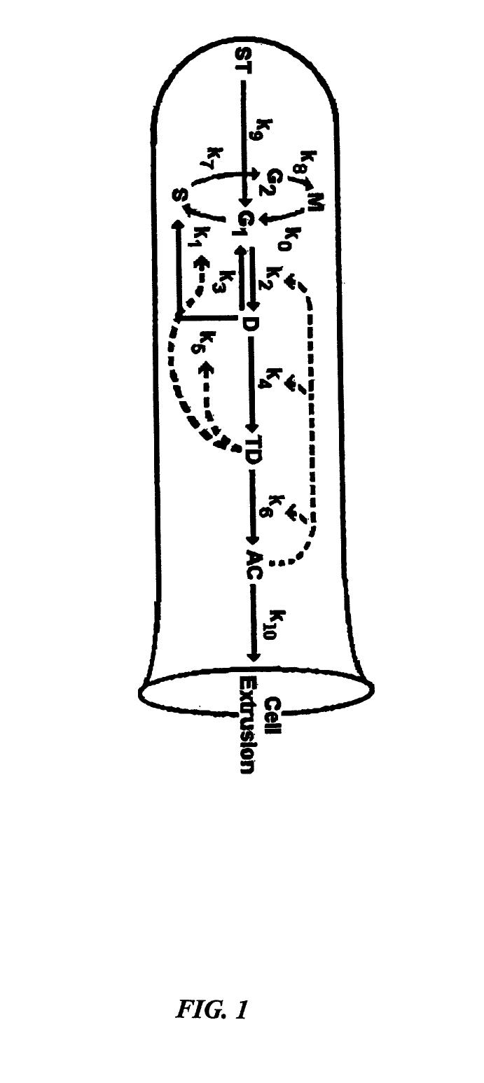

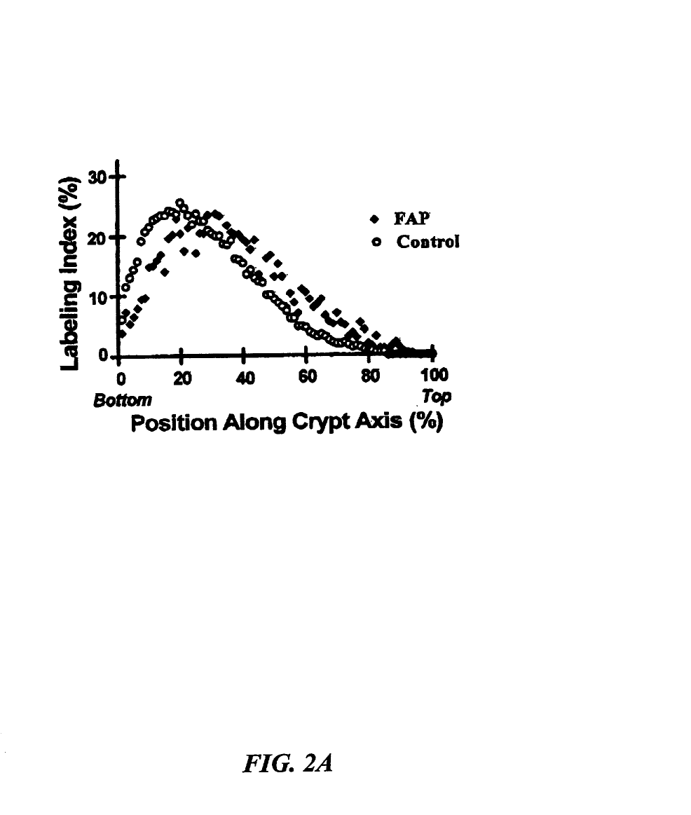

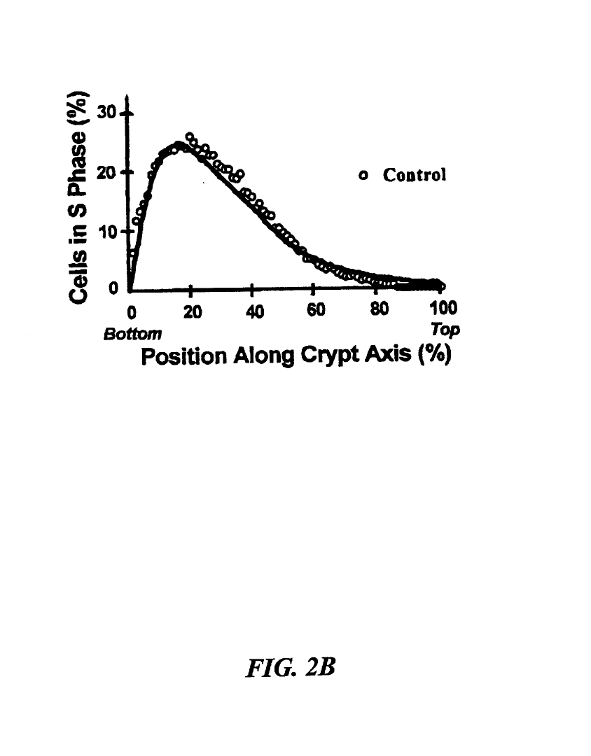

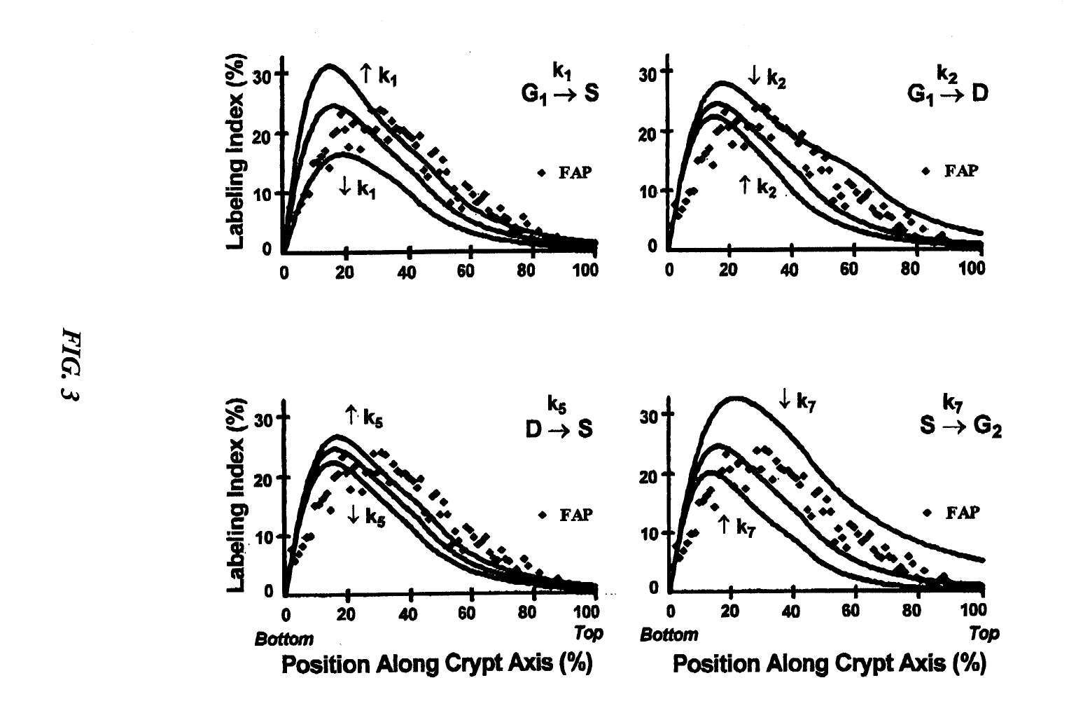

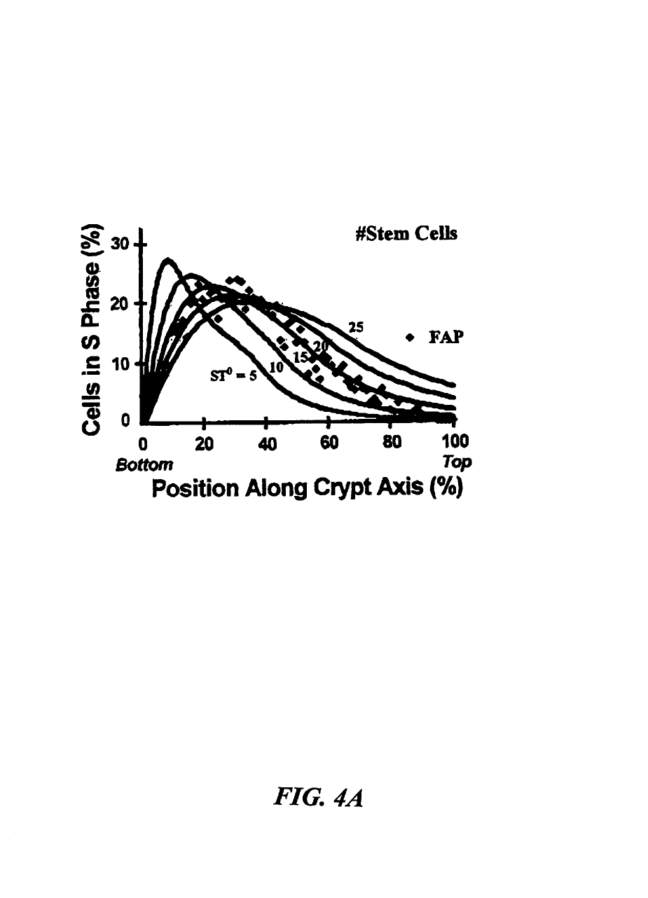

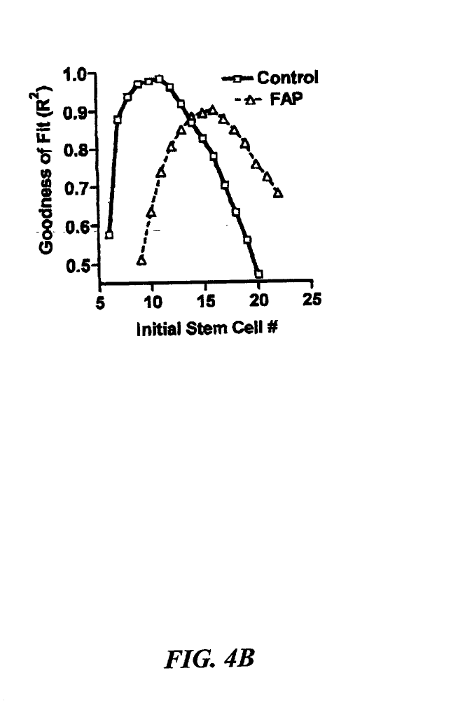

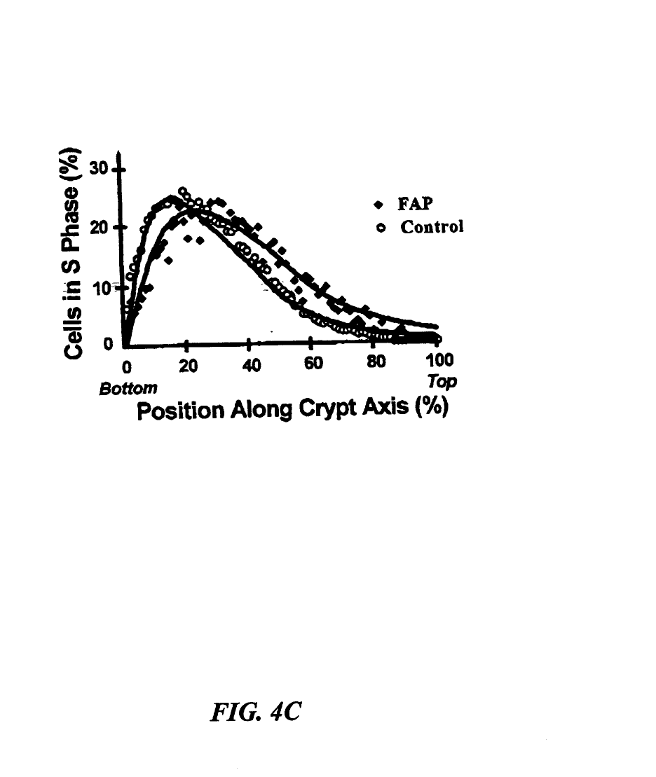

CPD Model Design. The scheme shown in FIG. 1, from which the computer model was constructed, can be viewed as having two parts within the main mechanism (solid arrows). Cellular proliferation (left-hand side of scheme) is a closed system specified by the cell cycle (G₁→S→G₂→M→G₁→ . . . ) (57) and is the “default” mechanism for generating cells within the system (1). Cellular differentiation and apoptosis (right-hand side of scheme) represents an open system in which cells exit the cell cycle by the differentiation/apoptosis pathway (G₁→D→TD→AC) (18, 58). Differentiated (D) cells and terminally differentiated (TD) cells represent the population of all differentiated cell types found in human crypts. The scheme also shows stem cells (ST) as the initial cell type for generation of G₁phase cells (ST→G₁), which simulates the biologic mechanism by which stem cells at the crypt base generate progeny cells for crypt cell renewal (59). ST and the differentiation pathways are coupled to the cell cycle at the G₁ phase (60). Also incorporated are pathways for de-differentiation of D cells (D→G₁) and proliferation of non-terminally differentiated cells (D→S) (57). Cell loss is depicted by exit of AC cells from the system, which simulates biological extrusion of apoptotic cells at the crypt lumen (61). The CPD Model design also includes two feedback loop mechanisms (dashed arrows in FIG. 1). In the negative feedback mechanism, the TD cell population regulates two steps, D→S and G₁→S. In the positive feedback mechanism, the apoptotic (AC) cell population regulates three steps, G₁→D, D→TD, and TD→AC. Parameters that were incorporated into the model included initial number of ST cells (ST⁰) and eleven rate constants that govern the rate of the various cell cycle and differentiation/apoptosis steps.

FIG. 2.

A. The biological data from FAP and control crypts. The biological data modified from those of Potten (7), are displayed as the percent of bromodeoxyuridine-labeled cells as a function of cell position along the crypt axis for both healthy unaffected human controls (open circles) and FAP patients (solid diamonds). Comparison of the FAP LI to control LI shows that the FAP proliferative abnormality (resulting from a germline APC mutation) involves a shift in the LI toward the crypt top. Specifically, the FAP LI as compared to the control LI shows a shift in the S-phase curve peak position from 20% to 31.25% along the crypt axis while a slight decrease was observed in the peak height from 25.58% to 23.65% S-phase cells. This proliferative abnormality does not appear to involve hyperproliferation because the total number of labeled cells in FAP crypts is not significantly increased (i.e. the FAP:control ratio for “area-under-the-curve” is 1.05).

B. “Best-Fit” Simulation of Control Biological Data. Results on “best-fit” CPD model simulation of the control LI are displayed (solid black line) as the percent of S-phase cells (Y-axis) versus cell crypt axis position (X-axis).

FIG. 3.

Attempts to simulate the FAP LI Biological Data by Perturbation of Rate Constant Values. The “best-fit” CPD model output for the control LI is the middle curve in each panel. The S phase profiles (upper and lower curves) resulting from perturbation of the rate constants k₁, k₂, k₅, and k₇ are also shown after a 50% increase or decrease in each parameter value. Perturbation of k₄ gave graphical output (not shown) that was nearly identical to results shown for perturbation of k₂. Perturbation of rate constants k₁, k₂, k₄, k₅, and k₇ failed to produce a S phase curve profile that fits the FAP biological data. Perturbation of k₀, k₃, k₆, k₈, k₉, and k₁₀ had no appreciable effect on the S-phase profile (data not shown).

FIG. 4

A. Shifting of the S phase curve along the crypt axis due to increasing initial number of stem cells (ST⁰). Perturbations of other model parameters failed to produce such a right-shift of the S phase curve. Numbers next to the curves indicate values for ST⁰.

B. Goodness of Fit of CPD Model Simulation with Control and FAP Biological Data. Goodness of Fit was calculated using nonlinear regression analysis (25, 26) based on a polynomial curve derived from the simulated output. The polynomial curve was then used to determine fit with the biological data. The R² is the fraction of the variation that is shared between X and Y, which ranges between 0 and 1.0 with 0 being an absence of fit and 1.0 being an exact fit.

C. “Best-Fit” Simulation of FAP and Control Biological Data. The figure shows “best-fit” CPD model simulation (solid black lines) in relation to the control and FAP biological data (data points).

### DETAILED DESCRIPTION

Mechanisms that cause normal tissues to become malignant involve an “enormously complex process” (1). Indeed, complexity in carcinogenesis occurs at each of many hierarchical levels. Even at the genetic level, tumor cells accumulate mutations in multiple genes during formation of most cancer types. Cancer is also the outcome of altered mechanisms occurring at other levels involving RNA, proteins, intracellular pathways, intercellular interactions, tissues, organs, individuals, families, society, etc. Since events occurring at one hierarchical level feed into and modify mechanisms at other levels, cancer development is a dynamic process that is more complicated than a simple summation of the parts.

Because of this interaction between different levels, conventional in vivo and in vitro studies have limitations in the study of cancer formation. In vivo studies are limited by their difficulty in identifying and/or controlling all of the variables in a reaction sequence whereas in vitro studies of independent isolated processes are limited in their ability to predict eventual outcome of interrelated multiple complex mechanisms. As a result of these limitations, little is known about how cancer-causing genetic mutations change the behavior of normal cells in a way that leads to alterations occurring at higher levels of complexity. Even in apparently simple situations, where an individual has a cancer-predisposing germline mutation, the relationship between this well-defined initiating event and development of the cancer phenotype at the tissue level is unknown. Why a germline cancer-predisposing mutation that is present in all cells of every tissue of the individual, leads only to the spectrum of specific tumor types that occurs in each hereditary cancer syndrome is also not understood. Consequently, elucidation of the mechanisms in complex processes such as cancer development will require a different experimental approach.

In this paper, a computer model (FIG. 1) is used to study the cellular mechanism that links a cancer predisposing germline mutation to the earliest known tissue change in the development of colorectal cancer (CRC). Theoretical interpretation of complex processes based on mathematical modeling has several advantages. First, modeling provides a defined and quantitative context in which different outcomes can be evaluated and compared following changes in one or more input parameters. Second, modeling can be used to quantitatively test the validity of proposed mechanisms. Finally, modeling provides a rapid and practical method to conduct “experiments” that cannot, because of system complexity, be accomplished by in vitro or in vivo approaches.

The tumorigenic process in one hereditary form of CRC, familial adenomatous polyposis (FAP), is an ideal system for a modeling study since both the initiating genetic event and earliest tissue change have been identified. The initiating genetic event in FAP tumors and in most sporadic CRC is a mutation in the adenomatous polyposis coli (APC) gene (2). The earliest known tissue change resulting from a germline APC mutation is an alteration in the distribution of DNA-synthesizing (S phase) cells in histologically normal-appearing colonic crypts of FAP patients. Specifically, in crypts of FAP patients, the labeling index (LI) based on tritium-labeled thymidine and bromodeoxyuridine uptake, when compared to control crypts from healthy unaffected individuals, displays a shift upwards, away from the crypt base and towards the crypt top (3-7). The S phase cell distribution (LI) profiles for control and FAP crypts, plotted as S phase cell percent vs. crypt axis position, are displayed in FIG. 2 (data of Potten et al (7)).

Even though the initiating genetic and tissue events in CRC development are characterized in FAP, the mechanism linking these events remains unknown. Four mechanisms have been suggested. Three invoke a change in the rate of cell cycle proliferation and/or differentiation as the cause of the abnormal crypt LI pattern. These include: 1) a loss of regulatory control that normally suppresses DNA synthesis during cell migration in the upper portions of the crypt (8-10), 2) a decrease in duration of the G₁ cell cycle phase (11), and 3) a decline in differentiation of epithelial cells in colonic crypts (12, 13). A fourth postulated mechanism is that the stem cell is the origin of CRC (14). For example, Moser and coworkers (15), by studying an animal model of FAP, found histological evidence of different cell lineages in intestinal adenomas and proposed that “tumorigenesis in Min/+mice may be initiated by a stem cell normally located at the base of the intestinal crypt”. To our knowledge, no experiments have been reported that critically address any of these four hypotheses.

In contrast, numerous experiments have helped define the cellular functions of APC. Functions associated with APC include a role in gene transcriptional regulation via the beta-catenin pathway, cell-cell adhesion, cell migration, apoptosis, cell cycle control, and microtubule cytoskeletal dynamics. Nonetheless, the mechanism that defines the connection between loss of any or all of these cellular functions of APC and the proliferative change as seen in FAP patients has not been established.

To investigate the mechanism that links the germline APC gene mutation and the proliferative shift in FAP crypts, and to understand adenoma development, we created a cellular proliferation/differentiation (CPD) model. The CPD model design simulates the cellular dynamics of the colonic crypt (FIG. 1). The model takes into account: (1) that cell proliferation, differentiation, and apoptosis occur continuously in the crypt (16-18), (2) that as epithelial cells migrate up the crypt column they change in their capacity for cell division and differentiation (19), and (3) that the crypt, even in FAP patients, represents a highly regulated steady-state system whereby a constant number of cells is maintained via a balance between cell generation in the lower part of the crypt and cell loss at the top of the crypt (7).

Rate constants for each step in the CPD model (see FIG. 1 and accompanying legend) and rate equations for the rate of change of each cell type population as a function of time were written (20). Computation was performed by numerical integration (21, 22) using an iteration method described previously for other models (23, 24). CPD Model output was graphically displayed as the percent of S-phase cell population (Y-axis) as a function of cell crypt axis position (X-axis). The biological data sets of Potten et al (7) for LI of control and FAP crypts (FIG. 2A) were used because these data appeared to be based on one of the most valid and thorough studies available.

To fit the control LI biological data (FIG. 2A) from healthy unaffected individuals, simulation was performed by systematically adjusting parameter values (rate constants and initial ST number [ST⁰]) and modifying the model design (e.g., adding feedback loops). Using this iterative approach, a set of parameter values (20) and a model design (FIG. 1) was achieved that generated output that fit with control LI data (FIG. 2B). “Goodness-of-fit” analysis (25, 26) showed an outstanding fit of simulation data to control biological data (R²=0.98).

Feedback loops were needed in order to provide output that fit the control LI data. The requirement for feedback loops in our model is consistent with several biological observations: (i) in vivo, the total number of crypt cells is normally maintained at a constant value suggesting that cellular feedback mechanisms function to regulate the rate of cell proliferation, (ii) the Tcf1 transcription factor acts, in murine intestinal epithelium, as a negative feedback regulator of β-catenin and Tcf4 target genes which encode proteins downstream from APC in its signaling pathway (27), and (iii) the murine CRC gene CDX2, which normally induces terminal differentiation of intestinal epithelial cells, induces its own expression through auto-regulation (28).

To test the four hypotheses (discussed above) regarding the proliferative shift in FAP crypts, it was determined whether perturbation of any single CPD model parameter gave an S phase curve that mimics this LI shift. When rate constant values that had fit best with the control LI (20) were individually increased or decreased, none of the resultant S phase profiles fit the FAP biological data (FIG. 3). Perturbations of k₀, k₃, k₆, k₈, k₉ and k₁₀ had virtually no effect on the S phase cell profile (but did affect other cell type profiles—data not shown). Perturbations of k₁, k₂, k₄, k₅ and k₇ caused either an increase or decrease in the peak height of the S phase curve and slight shifts in peak position along the crypt axis (FIG. 3). However, none of these perturbations yielded curves that mimicked the LI shift in FAP. Modifying the feedback loops also failed to simulate FAP LI data.

In contrast, when the initial ST number (ST⁰) was perturbed, the S phase curve showed major shifts along the crypt axis. FIG. 4A shows that a right-shift occurs when ST⁰ is increased from 10 to 15 that mimics the proliferative shift from control to FAP crypts (FIG. 2A). “Goodness-of-fit” analysis (25, 26) was used to quantitatively determine which initial stem cell number yielded model data output that best fit with the biological data. This analysis resulted in bell-shaped curves, the peaks of which indicated best fits with either control or FAP data (FIG. 4B). An ST⁰ of 11 yielded an S phase curve that best fit the control LI ( R²=0.98) whereas an ST⁰ of 16 yielded an S phase curve that best fit the FAP LI (R²=0.90). Higher and lower values of ST⁰ yielded S phase curves that had poorer Goodness-of-Fit values for FAP and control. The effect of this perturbation in ST⁰ (from 11 to 16) in the model suggests that an increase of about 50% in the number of stem cells in the crypt underlies the shift observed biologically from control to FAP LI (see FIG. 4C).

Although other computer models (29-33) have been developed to study intestinal crypt cellular dynamics, to our knowledge the CPD Model is the first designed to investigate the cellular etiology of the proliferative abnormality in FAP crypts. Our model also provided an experimental method to test previous hypotheses on the cellular etiology of the LI shift in FAP.

Experiments using the CPD Model provide some new and interesting conceptual insights into the proliferative abnormality in FAP. First, the fact that, by using the same model mechanism (FIG. 1), CPD simulation was able to fit both control and FAP LI data suggests that the proliferative abnormality in vivo does not require introduction of a new biological mechanism for crypt epithelial cell renewal. Second, the fact that an identical set of rate constant values gave the best fit with both control and FAP LI data (20) suggests that the proliferative abnormality is not due to a cellular mechanism that alters the rate of cell cycle proliferation and/or differentiation. Third, CPD Model experiments suggest that an expansion in the crypt stem cell population is sufficient to explain the observed proliferative abnormality in FAP.

CPD results on simulation of the FAP LI not only provides a cellular mechanism for tumor initiation in FAP, but it also provides evidence that crypt stem cell overproduction is: i) caused by a germline APC mutation, ii) a key event at the cellular level that links initiating events at the genetic level and tissue level in CRC tumorigenesis, and iii) involved in tumor initiation in sporadic CRC, which often involves acquisition of a somatic APC mutation early in its development (2). This model of CRC initiation via stem cell overproduction may be generally relevant and also explain the initiation of many other cancer types.

CPD modeling provides the basis of yet another hypothesis, namely, that APC normally functions to control the number of stem cells in the colonic crypt. While this regulatory property of APC has not yet been described, indirect biological evidence is consistent with this hypothesis. In the Tcf4 knockout mouse, germline inactivation of Tcf4 transcription factor leads to depletion of epithelial stem cell compartments in the small intestine (34). These results coupled with the fact that activation of Tcf4 is known to occur when APC is mutant (35, 36) support the hypothesis that APC does, indeed, control stem cell numbers. Based on these observations it would be predicted that the stem cell compartment is expanded in FAP crypts because germline APC mutation would lead to activation of Tcf4. This prediction is consistent with evidence from CPD modeling indicating stem cell compartment expansion in the FAP crypt.

How might APC normally regulate stem cell number and how might APC mutation lead to an increase in stem cell number? In normal crypts there is an APC expression gradient with intracellular APC concentration being highest at the crypt top and lowest at crypt bottom (37, 38). High intracellular APC concentration (crypt top) is associated with apoptosis and low cell anchorage, properties of extruded cells. Low intracellular APC concentration (crypt bottom) is associated with cellular immortality (anti-apoptosis) and high cell anchorage, properties of stem cells. Hence, the intracellular APC concentration gradient from crypt bottom to top parallels the transition in crypt cell properties from those of stem cells to those of extruded cells. Thus, it appears that low APC concentration in the normal crypt bottom allows a few cells to retain their stem cell properties of immortalization and anchorage. In the FAP patient with a germline APC mutation, there should also be a gradient in intracellular concentration of wild-type APC, but APC concentrations will be reduced by 50% at all points along the crypt axis. At the crypt bottom, this reduction in APC will lead to a few more cells having stem cell properties. Our modeling results suggest about 50% more stem cells.

If, indeed, APC controls stem cell numbers, then it follows that when the remaining wild-type APC allele is lost (i.e. the second hit according to Knudson's hypothesis (39)), a further increase in the number of stem cells will occur. Biologically, tumor cells in adenomatous polyps in humans and Min/+mice frequently have mutation of both APC alleles (40). Moreover, mutation of both APC alleles is sufficient for the growth of early colorectal adenomas in FAP patients (41). Therefore, a logical mechanism for adenoma development is further clonal expansion of the stem cell population due to a second hit in APC. Indeed, CPD modeling shows that increasing the initial number of stem cells beyond 16 causes an even greater shift in the S-phase profile towards the crypt top (FIG. 4a). This mechanism is consistent with other mechanisms proposed to explain histogenesis of aberrant crypts and micro-adenomas (13, 42-45).

The “stem cell overproduction” mechanism is also consistent with the stem cell model of tumor growth based on the concept of hierarchical proliferation (46, 47). The hierarchical concept holds that neoplasms have a cell-renewal hierarchy that is similar to normal tissues and tumors contain three types of cells: i) proliferating, self-renewing stem cells, ii) proliferating non-renewing transitional cells, and iii) non-proliferating, differentiated end cells (48). The hierarchical concept also proposes that although the stem cell component of tumors is a small subset within the total cell population, its expansion constitutes growth of tumors.

That cancer originates from stem cells is not a new concept (49, 50), particularly in relation to the origin of leukemias (51-54) and of teratomas (55, 56). Indirect evidence also supports a stem cell origin for solid tumors such as CRC. Since tumorigenesis in the colon is a relatively slow process, short-lived non-stem cell populations within crypts are considered an unlikely origin of CRC (14). Additionally, histological evidence from Min/+mice indicates that multiple differentiated cell types exist in intestinal adenomas, which suggests a stem cell origin for intestinal tumors (15). The present study provides a mechanism for how stem cells are involved in the origin of CRC.

CPD modeling has provided insight into the “enormous complexities” of tumorigenesis and has provided a theoretical foundation to understand carcinogenesis in the colon. Results from CPD Model experiments have also provided a theoretical basis for our “stem cell overproduction” hypothesis in CRC development. According to this hypothesis, seven steps promote conditions favoring adenoma formation: 1) mutation of one APC allele occurs in a stem cell, 2) the APC mutation leads to expansion of the crypt stem cell population, 3) the stem cell population expansion produces a S-phase cell population shift toward the crypt top, 4) the second wild-type allele is inactivated in a heterozygote stem cell, 5) the second “hit” gives rise to further expansion of the stem cell population, 6) the proliferative shift advances even further toward the crypt top, and 7) the expanded clone of stem cells produces even more non-stem proliferating, non-terminally differentiated, terminally differentiated, and apoptotic cells. The combination of steps 5, 6 and 7 leads to promotion of adenoma development and growth in the colon. Based on this view of tumorigenesis, effective colorectal cancer therapy and chemoprevention require elimination or control of mutant stem cell populations.

All publications, figures, patents and patent applications cited herein are hereby expressly incorporated by reference for all purposes to the same extent as if each was so individually denoted.

References and Notes

01. C. Sonnenschein, A. M. Soto, The Society Of Cells-Cancer And Control Of Cell Proliferation, (BIOS Scientific Publishers Ltd and Springer-Verlag, New York, 1999).

02. S. M. Powell, et al., Nature 359, 235 (1992).

03. E. E. Deschner, C. M. Lewis, M. Lipkin, J. Clin. Invest. 42, 1922 (1963).

04. H. Bleiberg, P. Mainguet, P. Galand, Gastroenterology 63, 240 (1972).

05. T. Iwana, J. Utsunomiya, J. Sasaki, Jpn. J. Surg. 7, 230 (1977).

06. M. Lipkin, et al., Cancer Res. 43, 1899 (1983).

07. C. S. Potten, M. Kellett, D. A. Rew, S. A. Roberts, Gut 33, 524 (1992).

08. A. P. Maskens, E. E. Deschner, J. Natl. Cancer Inst. 58, 1221 (1977).

09. M. Lipkin, In: Physiology Of The Gastrointestinal Tract., L R Johnson, Ed. (Raven Press, New York, 1981), pp. 145-168.

10. E. E. Deschner, M. Lipkin, In, Gastrointestinal Tract Cancer. M. Lipkin, R. A. Good, Eds. (Plenum Medical Book Co., New York, 1978) pp. 3-24,.

11. G. Biasco, M. P. Paganelli, M. Miglioli, L. Barbara, J. Cellular Biochemistry Supplement 16G, 73 (1992).

12. M. Lipkin, Cancer 34, 878 (1974).

13. E. E. Deschner, Amer. J. Gastroenterol. 77, 207 (1982).

14. J. D. Potter, J. Natl. Cancer Inst. 91, 916 (1999).

15. A. R. Moser, W. F. Dove, K. A. Roth, J. I. Gordon. J. Cell Biol. 116, 1517 (1992).

16. A. M. Shamsuddin, P. C. Phelps, B. F. Trump, Hum. Pathol. 13, 790 (1982).

17. G. L. Eastwood, In Colon: Structure and Function. L Bustos-Fernandez, Ed. (Plenum Medical Book Co., New York, 1983) pp. 1-16.

18. A. Bedi, et al., Cancer Res. 55, 1811 (1995).

19. C. S. Potten, M. Loeffler, Development 110, 1001, 1990.

20. CPD Model Rate Equations.

dM/dt=k₈·[G₂]−k₀[M]

dST/dt=−k₉·[ST]

dG₁/dt=k₉·[ST]+2k₀·[M]k₃·[D]−k₁·[G₁]/(1+[TD])−k₂·[G_{1]}(1+[AC]²)

dD/dt=k₂·[G₁](1+[AC]²)−k₃·[D]−k₄·[D](1+[AC]²)−k₅·[D]/(1+[TD])

dTD/dt=k₄·[D](1+[AC]²)−k₆·[TD](1+[AC]²)

dS/dt=k₁·[G₁]/(1+[TD])+k₅·[D]/(1+[TD])−k₇·[S]

dG₂/dt=k₇·[S]−k₈·[G₂]

dAC/dt=k₆·[TD](1+[AC]²)−k₁₀·[AC]

For each of the eight cell types (ST, G₁, S, G₂, M, D, TD and AC) a rate equation was written which expresses the rate of change of the population size for each cell type as a function of time. Also included are expressions for feedback loop mechanisms. In the negative feedback mechanism, rate constants k₁ and k₅ are controlled by the expression (1/(1+[TD])). In the positive feedback mechanism, rate constants k₂, k₄, and k₆ are controlled by the expression (1+[AC]²). Selection of the first set of rate constant values was, by necessity, arbitrary since rate constant values for steps shown in FIG. 1 have not yet been reported. The number of stem cells in murine colonic crypts has been reported to be 4-16 stem cells per crypt (19). Thus, an initial ST (ST⁰) value of 10 was chosen. Rate Constant Values (k₀ to k₁₀) that gave the best fit for both control and FAP biological data were, respectively: 0.05, 0.03, 0.025, 0.00625, 0.025, 0.025, 0.018, 0.0325, 0.025, 0.125, 0. Rate constants represent inverse relative time units.

21. Rate equation sets were solved by numerical integration with Mathematica equation-solving software [Mathematica Wolfram Research, Inc., Version 2.2, License # L2516-9472].

22. S. Wolfram, In: Mathematica: A System For Doing Mathematics By Computer, 2^{nd} Edition, (Addison-Wedley Publishing Co., Reading, Mass., 1991) pp. 696-699.

23. M. Head, N. L. Meryhew, O. A. Runquist, J. Lab. Clin. Med. 128, 61 (1996).

24. N. L. Meryhew, J. Mukherjee, M. S. Creager, O. A. Runquist, J. Lab. Clin. Med. 120, 111 (1992).

25. Quantitative goodness-of-fit assessments for S-phase cell population/crypt axis output compared to biological control and FAP LI data were done by regression analysis (Prism 2.01, GraphPad Software, San Diego, Calif). Regression analysis was accomplished by generating a seventh order polynomial for the simulated S-phase cell population/crypt axis data and then comparing the polynomial with biological LI data to yield sum-of-squares (SS), s_{y.x}, and R² values.

26. H. J. Motulsky, GraphPad Prism Users Guide, (GraphPad Software Inc, San Diego, 1999), pp 257-283.

27. J. Roose, et al., Science 285, 1923 (1999).

28. L. T. da Costa, et al., Oncogene 18, 5010 (1999).

29. S. Tsubouchi, Am. J. Anatomy 161, 239 (1981).

30. M. Loeffler, et al., Cell Tissue Kinet. 19, 627 (1986).

31. M. Loeffler, C. S. Potten, U. Paulus, J. Glatzer, S. Chwalinski S, Cell Tissue Kinet. 21, 247 (1988).

32. M. Loeffler, A. Birke, D. Winton, C. S. Potten, J. Theor. Biol. 160, 471 (1993).

33. S. R. Sipic, G. Zajicek, Comput. Biomed. Res. 26, 393 (1993).

34. V. Korinek, et al., Nature Genetics 19, 379 (1998).

35. P. J. Morin, et al., Science 21, 1787 (1997).

36. V. Korinek, et al., Science 275, 1784 (1997).

37. K. J. Smith, et al., Proc. Natl. Acad. Sci. USA 90, 2846 (1993).

38. I. Miyashiro, et al., Oncogene 11, 89 (1995).

39. A. G. Knudson Jr., Proc. Natl. Acad. Sci., USA 68, 820, 1971.

40. D. B. Levy, et al., Cancer Res. 54, 5953 (1994).

41. H. Lamlum, et al., Proc. Natl. Acad. Sci., USA 97, 2225 (2000).

42. A. P. Maskens, Gastroenterology 77, 1245 (1979).

43. H. S. Wasan, et al., J Pathol 185, 246 (1998).

44. M. Bjerknes, J. Theor. Biol. 178, 381 (1996).

45. T. Takayama, et al., N. Engl. J. Med. 339, 1277 (1998).

46. W. J. Mackillop, A. Ciampi, J. E. Till, R. N. Buick, J. Natl. Cancer Inst. 70, 9 (1983).

47. D. Bonnet, J. E. Dick. Nature Med 3, 730 (1997).

48. J. Kummermehr, K. R. Trott, In Stem Cells, C. S. Potten, Ed., (Academic Press, London, 1997) pp. 363-399.

49. G. B. Pierce et al. Cancer-A Problem In Developmental Biology. (Prentice Hall, New York, 1974), pp. 79-84.

50. G. B. Pierce. In The Biological Basis of Cancer, R. G. McKinnel, R. E. Parchment, A. O. Perantoni, G. B. Pierce, Eds., (Cambridge Univ. Press, Cambridge UK, 1998), pp. 39-47.

51. T. Lapidot, et al., Nature 367, 645 (1994).

52. L. E. Ailles, B. Gerhard, H. Kawagoe, D. E. Hogge, Blood 94, 1761 (1999).

53. P. J. Fialkow, J. W. Singer, Chronic Leukemia, 3^{rd} Ed, DeVita et. al., Eds., (J. B. Lippincott Co., Philadelphia, 1989) pp. 1836-1852.

54. M. D. Minden, In Hemopoietic Stem Cells, D. W. Golde, F. Takaku, Eds., (Dekker, New York, 1985) pp. 274-289.

55. L. C. Stevens, J. Natl. Cancer Inst. 38, 549 (1967).

56. G. B. Pierce, L. C. Stevens, P. K. Nakane, J. Natl. Cancer Inst. 39, 755 (1967).

57. R. Baserga, In Principles and Practices of Oncology, V. T. DeVita, Jr., S. Hellman, S. A. Rosenberg, Eds., (JP Lippincott Co., Philadelphia, 1993) pp.60-66.

58. R. L. Sparks, et al., Cell Tissue Kinet. 23, 71 (1990).

59. C. S. Potten, C. Booth, D. M. Pritchard, Int. J. Exp. Path. 78, 219 (1997).

60. R. E. Scott, D. L. Florine, J. J. Wille Jr., K. Yun, Proc. Natl. Acad. Sci. USA 79, 845 (1982).

61. D. A. Baron, D. H. Miller, J. Electro. Microsc. Tec. 16, 15 (1990).

What is claimed is:

1. A method for preventing or treating tumor in a subject comprising administering to said subject an effective amount of a chemopreventive or therapeutic agent that stops or reduces generation of mutant stem cells in vivo.

2. A method for preventing or treating tumor in a subject comprising administering to said subject an effective amount of a chemopreventive or therapeutic agent that averts or reverses progression of premalignant stem cells that have at least one genetic mutation.

3. A method for preventing or treating cancer in a subject comprising administering to said subject an effective amount of a chemopreventive or therapeutic agent that eliminates or controls premalignant stem cells and tumor stem cell populations.

4. The method of anyone of claims 1-3, wherein said chemopreventive or therapeutic agent acts on premalignant stem cells and tumor stem cells via intracellular, intercellular and tissue pathways that modulate the rate of premalignant or tumor stem cell proliferation.

5. The method of anyone of claims 1-3, wherein said chemopreventive or therapeutic agent acts on premalignant stem cells and tumor stem cells via intracellular, intercellular or tissue pathways that modulate the number of premalignant or tumor stem cells in vivo.

6. The method of anyone of claims 1-3, wherein said chemopreventive or therapeutic agent acts on premalignant stem cells and tumor stem cells via pathways involving growth factors, cytokines, and their receptors including those that function via autocrine, paracrine, and endocrine loops which control premalignant or tumor stem cells.

7. The method of anyone of claims 1-3, wherein said chemopreventive or therapeutic agent acts on premalignant stem cells and tumor stem cells via cell-to-cell or cell-to-matrix signaling pathways including epithelial stem cell-epithelial stem cell, epithelial stem cell-to-stromal cell, and stem cell-local extracellular matrix environmental interactions or communications.

8. The method of anyone of claims 1-3, wherein said chemopreventive or therapeutic agent acts on premalignant stem cells and tumor stem cells via biological pathways that function during development, which lead to the generation of premalignant or tumor stem cells during the development of a subject.

9. The method of anyone of claims 1-3, wherein said chemopreventive or therapeutic agent acts on premalignant stem cells and tumor stem cells via immunological pathways that control premalignant or tumor stem cells.

10. The method of anyone of claims 1-3, wherein said tumor is colon cancer.
